(19) United States
(12) Patent Application Publication
(10) Pub. No.: US 2021/0298672 A1
(43) Pub. Date: Sep. 30, 2021

(54) WEARABLE DISPLAY DEVICE

(71) Applicant: Samsung Display Co., Ltd., Yongin-si (KR)

(72) Inventors: Hwa Jung LEE, Yongin-si (KR); Kyung Jun LEE, Yongin-si (KR); Sang Hyun YOON, Yongin-si (KR); Han Wook CHOI, Yongin-si (KR)

(21) Appl. No.: 17/082,331

(22) Filed: Oct. 28, 2020

(30) Foreign Application Priority Data
Mar. 24, 2020 (KR) 10-2020-0035653

(51) Int. Cl. A61B 5/00; G06F 1/16; G16H 40/67; A61B 5/0205

(52) U.S. Cl. CPC A61B 5/6804; G06F 1/163; A61B 5/0261; A61B 5/7445; A61B 5/0205; G16H 40/67

(57) ABSTRACT

A wearable display device according to one or more embodiments of the present invention includes: a substrate; a first sensing unit on the substrate; and a display unit arranged on the first sensing unit and including a light emitting element that emits first light to a front side to display an image and emits second light to a rear side facing the substrate, the first sensing unit being configured to measure a bio-signal generated from a user's body using the second light reflected from the user's body.

### CROSS-REFERENCE TO RELATED APPLICATION

The application claims priority to and the benefit of Korean Patent Application No. 10-2020-0035653, filed on Mar. 24, 2020 in the Korean Intellectual Property Office, the entire content of which is hereby incorporated by reference.

### BACKGROUND

### 1. Field

Aspects of embodiments of the present invention relate to a wearable display device.

### 2. Discussion

Recently, wearable devices have been manufactured in various forms. Particularly, as a wearable device of a clothing type, interest in the wearable device in which a bio-signal of a user can be measured by being worn by the user and a health condition can be checked through the bio-signal is increasing.

### SUMMARY

According to an aspect of embodiments of the present invention, a wearable display device capable of measuring a bio-signal of a user using light emitted from a light emitting element included in a display unit and monitoring a health condition according to the measured bio-signal in real time through the display unit is provided.

A wearable display device according to one or more embodiments of the present invention includes: a substrate; a first sensing unit on the substrate; and a display unit arranged on the first sensing unit and including a light emitting element that emits first light to a front side to display an image and emits second light to a rear side facing the substrate, and the first sensing unit is configured to measure a bio-signal generated from a user's body using the second light reflected from the user's body.

In an embodiment, the wearable display device may further include a control unit arranged on the substrate and controlling the first sensing unit and the display unit. The first sensing unit may include a light receiving element to receive the second light reflected from the user's body and convert a measured value into an electrical signal. The control unit may include a measurement circuit electrically connected to the light receiving element to measure the bio-signal based on the electrical signal.

In an embodiment, the light receiving element may be arranged so as not to overlap the light emitting element in a plan view.

In an embodiment, the first sensing unit may further include a light blocking pattern including at least one opening exposing the substrate.

In an embodiment, the at least one opening may overlap the light emitting element in a plan view.

In an embodiment, the light blocking pattern may cover an upper surface of the light receiving element.

In an embodiment, the light emitting element may emit red light or green light as the second light.

In an embodiment, the first sensing unit may further include a first sensing electrode and a second sensing electrode that are electrically connected to the measurement circuit and are arranged to be spaced apart from each other. The measurement circuit may measure the bio-signal based on an amount of change in capacitance between the first sensing electrode and the second sensing electrode.

In an embodiment, the first sensing electrode and the second sensing electrode may be arranged so as not to overlap the light emitting element in a plan view.

In an embodiment, the first sensing unit may include: a base layer including a first portion arranged on the substrate and a second portion that is bent under the substrate; and a first sensing electrode and a second sensing electrode arranged on the second portion of the base layer and electrically connected to the measurement circuit.

In an embodiment, the first sensing electrode and the second sensing electrode may be exposed from the base layer. The measurement circuit may be configured to apply a current to the user's body through the first sensing electrode, detect a voltage at a portion in contact with the user's body through the second sensing electrode, and measure the bio-signal of the user's body based on a detected voltage.

In an embodiment, the measurement circuit may generate sensing data based on a measured bio-signal. The control unit may further include a processor to control the display unit to display an image including biometric information of the user's body based on the sensing data.

In an embodiment, the control unit may further include: a display driving circuit to drive the display unit under a control of the processor; and a battery unit to provide a power source for measuring the bio-signal to the measurement circuit, and to provide a power source for driving the display unit to the display driving circuit. The battery unit may be chargeable by solar energy charging or vibration energy charging.

In an embodiment, the wearable display device may further include a clothing unit formed of a material to be in close contact with the user's body, and the substrate may be attached to at least a portion of the clothing unit.

In an embodiment, the wearable display device may further include: a second sensing unit attached at a position where the substrate is not attached in the clothing unit; and a connecting member electrically connecting the measurement circuit and the second sensing unit. The measurement circuit may measure the bio-signal using the second sensing unit.

In an embodiment, the connecting member may be formed of a conductive fiber material.

In an embodiment, the wearable display device may further include: a second sensing unit attached at a position where the substrate is not attached in the clothing unit; and a communication unit to transmit a signal between the measurement circuit and the second sensing unit. The measurement circuit may measure the bio-signal using the communication unit and the second sensing unit.

### BRIEF DESCRIPTION OF THE DRAWINGS

The accompanying drawings, which are included to provide a further understanding of the inventive concepts, and are incorporated in and constitute a part of this specification, illustrate some example embodiments of the inventive concepts, and, together with the description, serve to explain principles of the inventive concepts.

FIG. 1 is a diagram illustrating a wearable display device according to one or more embodiments of the present invention.

FIG. 2 is a diagram illustrating an example of a bio-signal sensor included in the wearable display device of FIG. 1.

FIG. 3 is a diagram illustrating an example of a user bio-signal sensing operation of the bio-signal sensor of FIG. 2.

FIGS. 4A and 4B are diagrams illustrating one or more embodiments of the bio-signal sensor included in the wearable display device of FIG. 1.

FIG. 5 is a diagram illustrating an example of a user bio-signal sensing operation of the bio-signal sensor of FIGS. 4A and 4B.

### DETAILED DESCRIPTION

As the present invention allows for various changes and numerous embodiments, some example embodiments will be illustrated in the drawings and described in further detail in the written description. However, this is not intended to limit the present invention to particular modes of practice, and it is to be appreciated that all changes, equivalents, and substitutes that do not depart from the spirit and technical scope of the present invention are encompassed in the present invention.

Throughout the disclosure, like reference numerals refer to like parts throughout the various figures and embodiments of the present invention. The sizes of elements in the accompanying drawings may be exaggerated for clarity of illustration. It is to be understood that, although the terms “first,” “second,” etc. may be used herein to describe various elements, these elements should not be limited by these terms. These terms are used to distinguish one element from another element. For instance, a first element discussed below could be termed a second element without departing from the teachings of the present invention. Similarly, the second element could also be termed the first element. In the disclosure, singular forms are intended to include the plural forms as well, unless the context clearly indicates otherwise.

It is to be further understood that the terms “comprise,” “include,” “have,” etc. used in the disclosure, specify the presence of stated features, integers, steps, operations, elements, components, and/or combinations thereof, but do not preclude the presence or addition of one or more other features, integers, steps, operations, elements, components, and/or combinations thereof.

In addition, when an element is “coupled” to another element, this includes not only a case in which the element is directly coupled to the other element, but also a case in which one or more other elements are coupled therebetween.

Further, when a first part, such as a layer, a film, a region, or a plate is disposed on a second part, the first part may be not only directly on the second part but one or more third parts may be intervening therebetween. In addition, when a first part, such as a layer, a film, a region, or a plate is formed on a second part, a surface of the second part on which the first part is formed is not limited to an upper surface of the second part but may include other surfaces, such as a side surface or a lower surface of the second part. Similarly, when a first part, such as a layer, a film, a region, or a plate is under a second part, the first part may be not only directly under the second part but one or more third parts may be intervening therebetween.

Unless otherwise defined, all terms (including technical and scientific terms) used herein have the same meaning as commonly understood by one of ordinary skill in the art to which example embodiments of the inventive concept belong. It is to be further understood that terms, such as those defined in commonly-used dictionaries, should be interpreted as having a meaning that is consistent with their meaning in the context of the relevant art and will not be interpreted in an idealized or overly formal sense unless expressly so defined herein.

Herein, some example embodiments of the present invention will be described in further detail with reference to the accompanying drawings. The same or similar reference numerals are used for the same or similar components in the drawings.

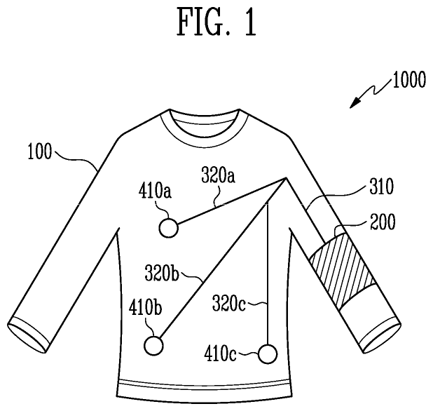

FIG. 1 is a diagram illustrating a wearable display device according to one or more embodiments of the present invention.

Referring to FIG. 1, a wearable display device 1000 may include a clothing unit 100 and a bio-signal sensor 200.

The clothing unit 100 may be formed of a material and a structure to be in close contact with a user's body. To this end, the clothing unit 100 may be made of a material having excellent elasticity. For example, the clothing unit 100 may be made of spandex material, various fiber materials in which spandex is mixed, or the like. As the clothing unit 100 is in close contact with the user's body, accuracy of bio-signal measurement by the bio-signal sensor 200 can be improved.

In FIG. 1, the clothing unit 100 is shown as a T-shirt (or top), but this is merely an example, and the clothing unit 100 is not limited thereto. For example, the clothing unit 100 may be composed of various wearable products, such as bottoms, shoes, socks, and protectors.

The bio-signal sensor 200 may be attached to at least a portion of the clothing unit 100. In FIG. 1, the bio-signal sensor 200 is shown as being attached to an arm portion of the clothing unit 100, but this is merely an example, and the present invention is not limited thereto. The bio-signal sensor 200 may be attached to another portion of the clothing unit 100. In addition, the bio-signal sensor 200 may be composed as a plurality and attached to the clothing unit 100.

The bio-signal sensor 200 may measure a bio-signal generated from the user's body, and may display an image including biometric information of the user based on the measured bio-signal. For example, the bio-signal sensor 200 may measure any of an electrocardiogram (ECG), respiration, activity, body temperature, and the like as the bio-signal.

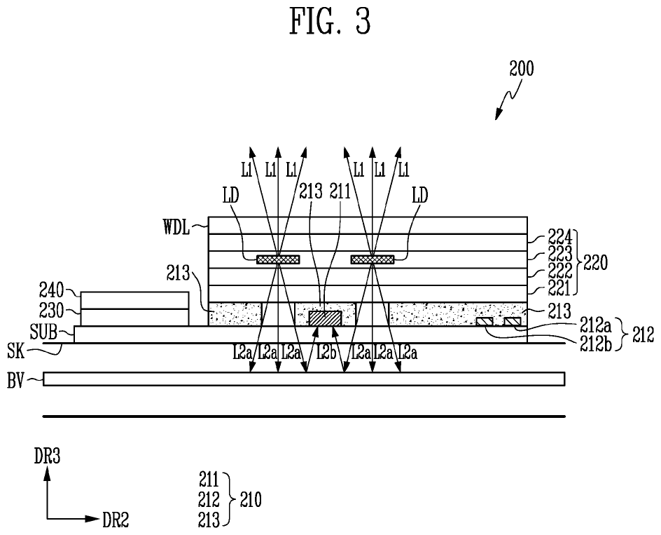

The bio-signal sensor 200 may include a sensing unit 210 (shown in FIG. 3) (or a first sensing unit) for measuring the bio-signal and a display unit 220 (shown in FIG. 3) for displaying the image including the biometric information of the user. Here, the display unit 220 (shown in FIG. 3) may include a light emitting element LD (shown in FIG. 3).

In an embodiment, the bio-signal sensor 200 may measure the bio-signal using light emitted from the light emitting element LD (shown in FIG. 3). For example, the light emitting element LD (shown in FIG. 3) may be composed of a double-sided light emitting element, and the bio-signal sensor 200 may measure the bio-signal generated from the user's body using light emitted from a rear surface (that is, a surface facing the user's body) of the display unit 220 (shown in FIG. 3) and reflected from the user's body. In addition, the bio-signal sensor 200 may display the image including the biometric information of the user on a front surface (that is, a surface viewed by the user) of the display unit 200 (shown FIG. 3). Accordingly, the user can monitor a health condition according to the measured bio-signal in real time.

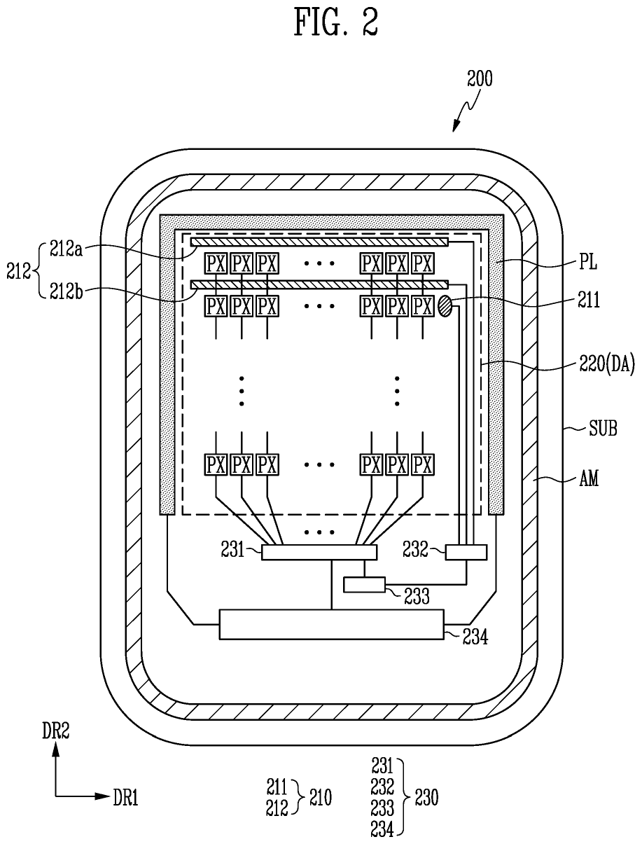

However, the image displayed by the bio-signal sensor 200 is not limited thereto. For example, the bio-signal sensor 200 may change the design of the clothing unit 100 (or wearable display device 1000) by displaying a logo, a specific pattern, and the like on the front surface of the display unit 200 (shown in FIG. 3). The bio-signal sensor 200 will be described further later with reference to FIGS. 2 and 3.

In an embodiment, the wearable display device 1000 may further include connecting members 310, 320a, 320b, and 320c and sensing units 410a, 410b, and 410c (or a second sensing unit). Here, the sensing units 410a, 410b, and 410c may be formed at different positions in a portion where the bio-signal sensor 200 is not attached. The connecting members 310, 320a, 320b, and 320c may electrically connect the bio-signal sensor 200 (or a measurement circuit 232 of FIG. 2) and the sensing units 410a, 410b, and 410c. Accordingly, the accuracy of bio-signal measurement by the user can be improved.

In an embodiment, the connecting members 310, 320a, 320b, and 320c may be made of a conductive fiber material for a feeling of wearing of the clothing unit 100. For example, the connecting members 310, 320a, 320b, and 320c may be configured in such a manner that a signal transmission line and a grounding line, which are made of conductive fibers, are disposed to be spaced apart from each other inside a fabric.

In FIG. 1, three sensing units 410a, 410b, and 410c are shown, but the number of sensing units 410a, 410b, and 410c is not limited thereto. For example, four or more sensing units may be provided. Accordingly, the accuracy of bio-signal measurement by the user can be improved.

In an embodiment, the wearable display device 1000 may not include the connecting members 310, 320a, 320b, and 320c. In this case, the wearable display device 1000 may further include a communication unit having a chip shape, for example, for transmitting a signal between the bio-signal sensor 200 and the sensing units 410a, 410b, and 410c such that the bio-signal sensor 200 measures the bio-signal of the user from the sensing units 410a, 410b, and 410c.

In an embodiment, the bio-signal sensor 200, the connecting members 310, 320a, 320b, and 320c, and the sensing units 410a, 410b, and 410c may be configured to be detachably attached to the clothing unit 100. Accordingly, when the user wears the wearable display device 1000, the bio-signal sensor 200, the connecting members 310, 320a, 320b, and 320c, and the sensing units 410a, 410b, and 410c may be attached to the clothing unit 100 to measure the bio-signal. In addition, when the user does not wear the wearable display device 1000, the bio-signal sensor 200, the connecting members 310, 320a, 320b, and 320c, and the sensing units 410a, 410b, and 410c may be detached from the clothing unit 100 to facilitate washing the clothing unit 100.

FIG. 2 is a diagram illustrating an example of a bio-signal sensor included in the wearable display device of FIG. 1; and FIG. 3 is a diagram illustrating an example of a user bio-signal sensing operation of the bio-signal sensor of FIG. 2.

Referring to FIG. 2, the bio-signal sensor 200 may include a display area DA in which the image is displayed. Pixels PX for displaying the image may be positioned in the display area DA. In an embodiment, each of the pixels PX may emit red, green, or blue light. Each pixel PX may include a light emitting element LD. For example, each pixel PX may include an organic light emitting diode.

The bio-signal sensor 200 may include a substrate SUB, the sensing unit 210 (or the first sensing unit), the display unit 220, and a control unit 230.

The substrate SUB may be in contact with (or in close contact with) a skin SK of a portion of the user's body where the bio-signal sensor 200 is attached. In an embodiment, the substrate SUB may be a flexible substrate capable of bending, folding, rolling, and the like to facilitate contact (or close contact) with the user's skin SK. For example, the substrate SUB may be formed of a polymer, such as polyimide (PI), polyacrylate (PA), or polyethylene terephthalate (PET).

Although not shown in FIGS. 2 and 3, the substrate SUB may include a flexible printed circuit board (FPCB) such that a signal is transmitted between the sensing unit 210 and the control unit 230 and a signal is transmitted between the display unit 220 and the control unit 230.

As described with reference to FIG. 1, the bio-signal sensor 200 may be detachably attached to the clothing unit 100. To this end, the bio-signal sensor 200 may further include an adhesive member AM disposed on a surface of the substrate SUB (for example, a surface contacting the user's skin SK).

The control unit 230 may control the sensing unit 210 and the display unit 220, and, in an embodiment, may include a display driving circuit 231, a measurement circuit 232, a processor 233, and a battery unit 234. In an embodiment, the display driving circuit 231, the measurement circuit 232, the processor 233, and the battery unit 234 included in the control unit 230 may be disposed so as not to overlap the display area DA on the substrate SUB.

The processor 233 may control the overall operation of the bio-signal sensor 200. For example, the processor 233 may be implemented as a system-on-chip.

The processor 233 may control the display driving circuit 231. The processor 233 may generate input image data and control signals, and provide the generated input image data and control signals to the display driving circuit 231.

The processor 233 may control the measurement circuit 232. The processor 233 may provide a control signal for measuring the bio-signal of the user to the measurement circuit 232.

Further, the processor 233 may include a memory, a communication device, and the like.

The display driving circuit 231 may drive the display unit 220 under the control of the processor 233. The display driving circuit 231 may generate a data signal and a scan signal based on the input image data and the control signals provided from the processor 233. In addition, the display driving circuit 231 may supply the data signal to each of the pixels PX through data lines, and supply the scan signal to each of the pixels PX through scan lines.

In an embodiment, the display driving circuit 231 may be formed of an integrated circuit (IC) and may be attached on the substrate SUB by a chip on glass (COG) method, a chip on plastic (COP) method, or an ultrasonic bonding method.

The measurement circuit 232 may measure the bio-signal of the user under the control of the processor 233. The measurement circuit 232 may be electrically connected to a light receiving element 211 or sensing electrodes 212 included in the sensing unit 210 to measure the bio-signal of the user. The measurement circuit 232 may generate sensing data based on the measured bio-signal of the user and provide the sensing data to the processor 233.

In an embodiment, the processor 233 may control the display unit 220 to display the image including the biometric information of the user based on the sensing data provided from the measurement circuit 232. To this end, the processor 233 may generate the input image data corresponding to a user biometric information image and provide the generated input image data to the display driving circuit 231.

The battery unit 234 may provide a power source for measuring (e.g., required for measuring) the bio-signal in the measurement circuit 232 to the measurement circuit 232, and a power source for driving (e.g., required for driving) the display unit 220 in the display driving circuit 231 to the display driving circuit 231.

In an embodiment, the battery unit 234 may be charged by a solar energy charging method or a vibration energy charging method. To this end, in an embodiment, the bio-signal sensor 200 may include a charging module 240 formed on the control unit 230. For example, the charging module 240 may be configured as a solar charging module or a vibration charging module.

Power source lines PL for supplying power sources for driving (e.g., required for driving) the pixels PX may be formed outside the display area DA. The battery unit 234 may supply the power sources for driving the pixels PX through the power source lines PL. In an embodiment, the power source lines PL may include two lines formed in a second direction DR2 outside the display area DA and one line formed in a first direction DR1 outside the display area DA.

The sensing unit 210 may be disposed on the substrate SUB, and may include the light receiving element 211, the sensing electrodes 212, and a light blocking pattern 213.

In an embodiment, the light receiving element 211 may be disposed so as not to overlap the pixels PX (or light emitting elements LD included in the pixels PX). Accordingly, a path through which the light emitted from the pixels PX moves to a surface of the user's skin SK may be formed.

In FIGS. 2 and 3, one light receiving element 211 is shown; however, this is merely an example. Two or more light receiving element 211 may be applied. Accordingly, the accuracy of bio-signal measurement by the bio-signal sensor 200 can be improved.

In an embodiment, a first sensing electrode 212a and a second sensing electrode 212b may be disposed so as not to overlap the pixels PX (or the light emitting elements LD included in the pixels PX) along the first direction DR1. Accordingly, a path through which the light (or second light L2a) emitted from the pixels PX moves to the surface of the user's skin SK may be formed.

The first sensing electrode 212a and the second sensing electrode 212b may be disposed to be spaced apart from (or so as not to contact) the user's skin SK by the substrate SUB. Capacitance may be formed between the first sensing electrode 212a and the second sensing electrode 212b disposed to be spaced apart from each other. The capacitance formed between the first sensing electrode 212a and the second sensing electrode 212b may be changed due to the influence of the bio-signal of the user. Here, the sensing electrodes 212 may be composed of at least two or more to form the capacitance.

In an embodiment, the bio-signal sensor 200 (or the measurement circuit 232) may measure the bio-signal based on the amount of change in the capacitance between the first sensing electrode 212a and the second sensing electrode 212b.

For example, when the bio-signal sensor 200 is in contact with the user's body (or user's skin SK), a capacitance value between the first sensing electrode 212a and the second sensing electrode 212b at the contact area may be changed minutely under the influence of the bio-signal of the user. The measurement circuit 232 may measure the amount of change in the capacitance value, amplify the amount of change in the measured capacitance value through an amplifier (AMP), and filter the information not related to the bio-signal to measure the bio-signal. As described above, the measurement circuit 232 may generate the sensing data based on the measured bio-signal and provide the sensing data to the processor 233. The processor 233 may control the display unit 220 to display the image including the biometric information of the user based on the sensing data provided from the measurement circuit 232.

In FIGS. 2 and 3, two sensing electrodes 212 are shown; however, this is merely an example. Three or more sensing electrodes 212 may be applied. Accordingly, the accuracy of the bio-signal measurement by the bio-signal sensor 200 may be improved.

The light blocking pattern 213 may be disposed on the substrate SUB, and may serve as a black matrix (BM) blocking the light emitted from the light emitting element LD. In an embodiment, the light blocking pattern 213 may be formed of a black organic polymer material including black dye or pigment capable of blocking light, or a metal (or a metal oxide), such as chromium or chromium oxide.

In an embodiment, the light blocking pattern 213 may include at least one opening exposing the substrate SUB. Since the light blocking pattern 213 includes the opening, a path through which the light (or the second light L2a) emitted from the pixels PX moves to the surface of the user's skin SK through the opening and a path through which the light (or second light L2b) reflected from the user's skin SK moves to the light receiving element 211 may be formed.

In an embodiment, the light blocking pattern 213 may cover an upper surface (for example, a surface facing the display unit 220) of the light receiving element 211. Accordingly, a path through which the light (or the second light L2a) emitted from the pixels PX directly moves to the light receiving element 211 without passing through the surface of the user's skin SK may be blocked.

The display unit 220 may be disposed on the sensing unit 210, and may include a pixel circuit layer 221, a first electrode layer 222, a light emitting element layer 223, and a second electrode layer 224.

The pixel circuit layer 221 may be disposed on the sensing unit 210. The scan lines, the data lines, and the like, as well as transistors of each of the pixels PX, may be disposed in the pixel circuit layer 221. Each of the transistors may include a gate electrode, a semiconductor layer, a source electrode, and a drain electrode.

The first electrode layer 222, the light emitting element layer 223, and the second electrode layer 224 may be sequentially stacked on the pixel circuit layer 221 along a third direction DR3. The light emitting element layer 223 may include the pixels PX that emit the light and a pixel defining layer that defines the pixels PX. The pixels PX of the light emitting element layer 223 may be disposed in the display area DA.

In an embodiment, the light emitting element layer 223 may be an organic light emitting layer including an organic material. In this case, the light emitting element layer 223 may include a hole transporting layer, the organic light emitting layer, and an electron transporting layer.

In an embodiment, the light emitting element layer 223 may include an inorganic light emitting element. In this case, the first electrode layer 222 and the second electrode layer 224 may be disposed on the same layer, and the inorganic light emitting element may be electrically connected to a first electrode of the first electrode layer 222 and a second electrode of the second electrode layer 224.

In an embodiment, the light emitting element layer 223 may include the double-sided light emitting element LD. The light emitting element LD may emit first light L1 to the front surface (for example, the surface viewed by the user), and emit the second light L2a to the rear surface (for example, the surface facing the sensing unit 210 or the user's skin SK).

The display unit 220 may display the image on the front surface using the first light L1 emitted from the light emitting element LD. For example, the display unit 220 may display the image including the biometric information of the user using the first light L1. Accordingly, the user can monitor the health condition according to the measured bio-signal in real time through the display unit 220.

In an embodiment, the bio-signal sensor 200 (or the sensing unit 210) may measure the bio-signal generated from the user's skin SK using the second light L2b emitted from the light emitting element LD to the rear surface (for example, the surface facing the sensing unit 210 or the user's skin SK) and reflected from the user's body (for example, a user's blood vessel BV).

In an embodiment, the light receiving element 211 may receive the second light L2b emitted from the light emitting element LD to the rear surface and reflected from the user's body (for example, the user's blood vessel BV), and convert the measured value into an electrical signal (for example, a current or the like).

In an embodiment, the light receiving element 211 may measure an amount of change in intensity of the reflected light according to an amount of blood flow in the user's blood vessel BV. For example, at a systolic peak in which the amount of blood flow increases due to the contraction of the heart, the blood pressure increases, but the intensity of the reflected light may be weak because the light (or the second light L2a) is absorbed by the blood to a relatively large degree. In addition, the intensity of the reflected light in a diastolic phase may be strong. In this way, the light receiving element 211 may measure the amount of change in the intensity of the reflected light and convert the measured value into the electrical signal. The light receiving element 211 may be formed of, for example, a photodiode (PD). The light receiving element 211 may provide the converted electrical signal to the measurement circuit 232.

In an embodiment, the bio-signal sensor 200 may use green light in a wavelength range of 530 nm that can be used as a neutral light source for correcting the amount of pulse wave or signal in consideration of light absorption as the second light L2a for measuring the bio-signal of the user. In another embodiment, the bio-signal sensor 200 may use red light in a wavelength range of 660 nm that can be used to obtain pulse waves for measuring the blood pressure as the second light L2a for measuring the bio-signal of the user. That is, in an embodiment, in order to measure the bio-signal of the user, the light emitting element LD may emit the red light or the green light as the second light L2a.

The measurement circuit 232 may measure the bio-signal based on the electrical signal provided from the light receiving element 211. For example, the measurement circuit 232 may include a trans-impedance amplifier (TIA), a programmable gain amplifier (PGA), and an analog-to-digital converter (ADC) for signal processing. Here, the trans-impedance amplifier may convert the electrical signal (for example, current) into an output voltage, the programmable gain amplifier may amplify a gain value of the output voltage, and the analog-to-digital converter may convert an analog output voltage value to a digital value to generate the bio-signal.

As described above, the measurement circuit 232 may generate the sensing data based on the measured bio-signal and provide the sensing data to the processor 233. The processor 233 may control the display unit 220 to display the image including the biometric information of the user based on the sensing data provided from the measurement circuit 232.

In an embodiment, the bio-signal sensor 200 may further include a window layer WDL disposed on the display unit 220. The window layer WDL may be disposed on the sensing unit 210 and the display unit 220 to protect the sensing unit 210 and the display unit 220 from external scratches or the like. A front surface (or a top surface) of the window layer WDL may be a surface in contact with a user's input means (e.g., a finger).

In an embodiment, the bio-signal sensor 200 may further include a polarizing layer (not shown) disposed between the display unit 220 and the window layer WDL. In an embodiment, the window layer WDL may be attached on the display unit 220 and the polarizing layer by an adhesive layer. Here, an optical clear adhesive (OCA) or an optical clear resin (OCR) may be used as the adhesive layer.

The substrate SUB and the window layer WDL may be formed to be transparent such that light travel paths of the first light L1 and the second light L2a and L2b are formed.

As described with reference to FIGS. 1 to 3, the wearable display device 1000 (or the bio-signal sensor 200) according to embodiments of the present invention may include the sensing unit 210 capable of measuring the bio-signal using the second light L2a emitted from the light emitting element LD of the display unit 220. Accordingly, since the sensing unit 210 does not include a separate light source for measuring the bio-signal, the configuration of the wearable display device 1000 can be simplified.

In addition, the wearable display device 1000 according to embodiments of the present invention may include the display unit 220 that displays the image including the biometric information of the user. Accordingly, the user can monitor the health condition according to the measured bio-signal in real time through the display unit 220.

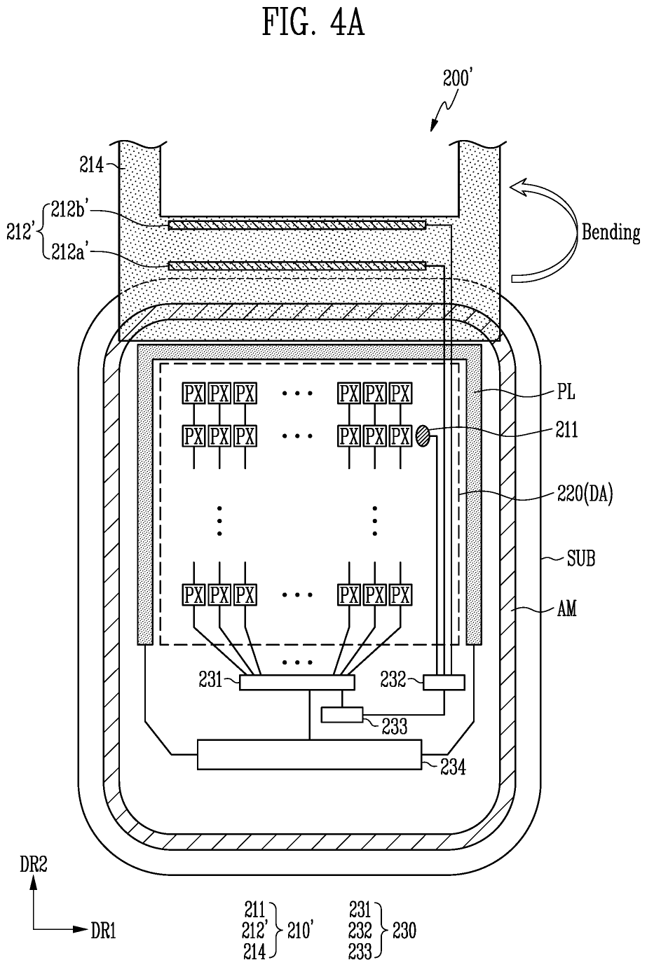

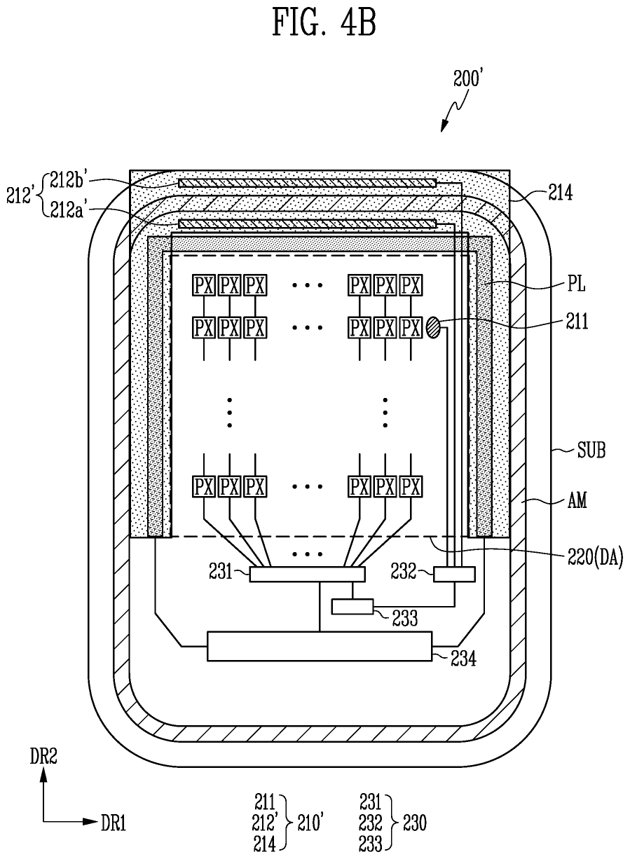

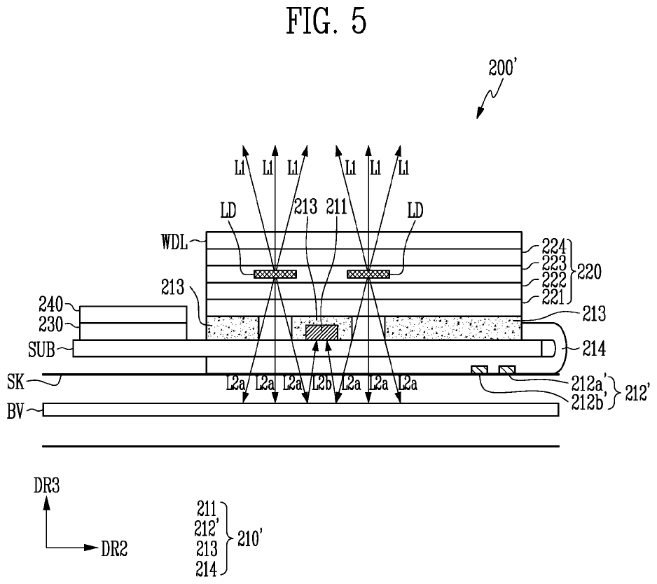

FIGS. 4A and 4B are diagrams illustrating one or more embodiments of the bio-signal sensor included in the wearable display device of FIG. 1; and FIG. 5 is a diagram illustrating an example of a user bio-signal sensing operation of the bio-signal sensor of FIGS. 4A and 4B. In FIGS. 4A, 4B, and 5, except for a configuration in which a bio-signal sensor 200′ further includes a base layer 214 and a configuration in which sensing electrodes 212′ are formed on the base layer 214, the bio-signal sensor 200′ may be substantially the same or similar to the bio-signal sensor 200 of FIGS. 2 and 3. Therefore, redundant descriptions may be omitted.

Referring to FIGS. 4A, 4B, and 5, the sensing unit 210 may further include the base layer 214.

The base layer 214 may include a first sensing electrode 212a′ and a second sensing electrode 212b′ electrically connected to the measurement circuit 232.

In an embodiment, a first portion of the base layer 214 (for example, one end of the base layer 214) may be disposed on the substrate SUB. In addition, the base layer 214 may be bent to a lower surface (for example, a surface facing the user's body) of the substrate SUB, such that a second portion of the base layer 214 (for example, the other end of the base layer 214) and the sensing electrodes 212′ may be disposed on the lower surface of the substrate SUB. To this end, in an embodiment, the base layer 214 may be formed of a flexible printed circuit board (FPCB).

The first sensing electrode 212a′ and the second sensing electrode 212b′ may be exposed from the base layer 214 while the base layer 214 is bent. Accordingly, the first sensing electrode 212a′ and the second sensing electrode 212b′ may contact the user's body (for example, the user's skin SK).

In an embodiment, the measurement circuit 232 may measure the bio-signal of the user using the first sensing electrode 212a′ and the second sensing electrode 212b′. For example, the measurement circuit 232 may apply a current to the user's body through one of the sensing electrodes 212′ (or a current electrode) (for example, the first sensing electrode 212a′ or the second sensing electrode 212b′). In an embodiment, the measurement circuit 232 may detect a voltage at a portion in contact with the user's body through the other of the sensing electrodes 212′ (or a voltage electrode) (for example, the second sensing electrode 212b′ or the first sensing electrode 212a′). In an embodiment, the measurement circuit 232 may calculate an impedance value corresponding to the user using the amount of current applied through the current electrode and the voltage measured through the voltage electrode. The measurement circuit 232 may generate the sensing data based on the impedance value.

The wearable display device according to embodiments of the present invention may include the sensing unit capable of measuring the bio-signal using the light emitted from the display unit. Accordingly, since the sensing unit does not include a separate light source for measuring the bio-signal, the configuration of the wearable display device can be simplified.

In addition, according to the wearable display device according to embodiments of the present invention, the user can monitor a health condition according to the measured bio-signal in real time through the display unit.

However, aspects and effects of the present invention are not limited to those described above, and may be variously extended without departing from the spirit and scope of the present invention.

The foregoing detailed descriptions may illustrate and describe the present invention according to some example embodiments. However, the foregoing descriptions merely illustrate and describe some example embodiments of the present invention. As described above, the present invention may be used in various different combinations, modifications and environments, and may be changed or modified within the scope of the inventive concept disclosed in this specification, the scope equivalent to the above-described description, and/or the scope of technology or knowledge of the art. Therefore, the description is not intended to limit the disclosure to the form disclosed herein. Also, it is intended that the appended claims be construed to include alternative embodiments.

What is claimed is:

1. A wearable display device comprising:
a substrate;
a first sensing unit on the substrate; and
a display unit arranged on the first sensing unit and comprising a light emitting element that emits first light to a front side to display an image and emits second light to a rear side facing the substrate,
wherein the first sensing unit is configured to measure a bio-signal generated from a user's body using the second light reflected from the user's body.

2. The wearable display device of claim 1, further comprising:
a control unit arranged on the substrate and controlling the first sensing unit and the display unit,
wherein the first sensing unit comprises a light receiving element to receive the second light reflected from the user's body and convert a measured value into an electrical signal, and
wherein the control unit comprises a measurement circuit electrically connected to the light receiving element to measure the bio-signal based on the electrical signal.

3. The wearable display device of claim 2, wherein the light receiving element is arranged so as not to overlap the light emitting element in a plan view.

4. The wearable display device of claim 2, wherein the first sensing unit further comprises a light blocking pattern comprising at least one opening exposing the substrate.

5. The wearable display device of claim 4, wherein the at least one opening overlaps the light emitting element in a plan view.

6. The wearable display device of claim 4, wherein the light blocking pattern covers an upper surface of the light receiving element.

7. The wearable display device of claim 2, wherein the first sensing unit further comprises a first sensing electrode and a second sensing electrode that are electrically connected to the measurement circuit and are arranged to be spaced apart from each other, and
wherein the measurement circuit measures the bio-signal based on an amount of change in capacitance between the first sensing electrode and the second sensing electrode.

8. The wearable display device of claim 7, wherein the first sensing electrode and the second sensing electrode are arranged so as not to overlap the light emitting element in a plan view.

9. The wearable display device of claim 2, wherein the first sensing unit comprises:
a base layer comprising a first portion arranged on the substrate and a second portion that is bent under the substrate; and
a first sensing electrode and a second sensing electrode arranged on the second portion of the base layer and electrically connected to the measurement circuit.

10. The wearable display device of claim 9, wherein the first sensing electrode and the second sensing electrode are exposed from the base layer, and
wherein the measurement circuit is configured to apply a current to the user's body through the first sensing electrode, detect a voltage at a portion in contact with the user's body through the second sensing electrode, and measure the bio-signal of the user's body based on a detected voltage.

11. The wearable display device of claim 2, wherein the measurement circuit generates sensing data based on a measured bio-signal, and
wherein the control unit further includes a processor to control the display unit to display the image including biometric information of the user's body based on the sensing data.

12. The wearable display device of claim 11, wherein the control unit further comprises:
a display driving circuit to drive the display unit under a control of the processor; and
a battery unit to provide a power source for measuring the bio-signal to the measurement circuit, and to provide a power source for driving the display unit to the display driving circuit, and
wherein the battery unit is chargeable by solar energy charging or vibration energy charging.

13. The wearable display device of claim 2, further comprising:
a clothing unit formed of a material to be in close contact with the user's body,
wherein the substrate is attached to at least a portion of the clothing unit.

14. The wearable display device of claim 13, further comprising:
a second sensing unit attached at a position where the substrate is not attached to the clothing unit; and
a connecting member electrically connecting the measurement circuit and the second sensing unit,
wherein the measurement circuit measures the bio-signal using the second sensing unit.

15. The wearable display device of claim 14, wherein the connecting member is formed of a conductive fiber material.

16. The wearable display device of claim 13, further comprising:
a second sensing unit attached at a position where the substrate is not attached to the clothing unit; and
a communication unit to transmit a signal between the measurement circuit and the second sensing unit,
wherein the measurement circuit measures the bio-signal using the communication unit and the second sensing unit.

17. The wearable display device of claim 1, wherein the light emitting element emits red light or green light as the second light.
